(19) United States
(12) Patent Application Publication
(10) Pub. No.: US 2023/0414894 A1
(43) Pub. Date: Dec. 28, 2023

(54) Improvements to Adhesive Strips that Secure Nasal Pillows to Nostrils

(71) Applicant: Nozeseal, LLC, College Station, TX (US)

(72) Inventors: Mark T. Holtzapple, College Station, TX (US); Carol Gaskamp, Brenham, TX (US); Scott Brauer, Dripping Springs, TX (US)

(73) Assignee: Nozeseal, LLC, College Station, TX (US)

(21) Appl. No.: 18/255,786

(22) PCT Filed: Dec. 1, 2021
(86) PCT No.: PCT/US2021/061340

§ 371 Date: Jun. 2, 2023

Related U.S. Application Data
(60) Provisional application No. 63/121,816, filed on Dec. 4, 2020.

(51) Int. Cl. A61M 16/06

(52) U.S. Cl. CPC A61M 16/0688; A61M 16/0666

(57) ABSTRACT

Improved embodiments of the disclosure provide a universal sealing apparatus for one or more cannulas. The apparatus includes a first portion, a second portion, and a third portion. The first and second portions are configured to removably adhere directly or indirectly to a nose. The third portion is coupled to the first and second portions. The third portion removably couples to one or more cannulas and places a force upon a base of flared portions of the one or more cannulas when the first and second portions are adhered to the nose. The force maintains a fluid seal of the one or more cannulas within one or more respective nostrils of the nose. The third portion includes a reinforcement. In one configuration, the reinforcement is angled. In another configuration, the reinforcement has a hinge to allow easy adjustment on a nose. In yet another configuration, the reinforcement has slitted holes for cannulas. In yet another configuration, third portion is removably coupleable to the first and second portions.

### CROSS-REFERENCE TO RELATED APPLICATIONS

This application is a national stage filing of PCT Application No. PCT/US21/61340, which was filed on Dec. 1, 2021. PCT/US21/61340 claims priority to U.S. Patent Application No. 63/121,816 filed on Dec. 4, 2020 entitled “Improvements to Adhesive Stripes that Secure Nasal Pillows to Nostrils.” Both applications are hereby incorporated by reference in their entirety.

This application is an improvement of Applicant's prior patent, U.S. Pat. No. 10,238,827, which is hereby incorporated by reference for context.

### TECHNICAL FIELD

Aspects of the present disclosure relate to positive airway pressure (PAP) devices and, in particular, to improvements to a nasal pillow sealing device.

### BACKGROUND

Breathing-assist devices are used in a variety of situations where normal breathing functions are compromised, such as patients suffering from obstructive sleep apnea or those with compromised lung function resulting from weakened muscles, damaged nerves, or stiffened lung tissues. Lung function can be compromised by disease (e.g., pneumonia, flu, bronchitis), restriction of airways (e.g., asthma), muscle degeneration, nerve degeneration, genetic maladies (e.g., cystic fibrosis), drugs (e.g., opioids), exposure to smoke or chemicals, cancer, or chest injuries. Furthermore, breathing-assist devices may be used temporarily during surgery when anesthetics are administered.

In some cases, breathing maladies can be addressed simply by enriching normal atmospheric-pressure air with oxygen. In other cases, it is necessary to pressurize air to inflate the breathing passages. When breathing passages must be inflated, it is necessary to create a seal between the breathing passages and the tubes that deliver pressurized air.

### SUMMARY

Improved embodiments of the disclosure provide a universal sealing apparatus for one or more cannulas. The apparatus includes a first portion, a second portion, and a third portion. The first and second portions are configured to removably adhere directly or indirectly to a nose. The third portion is coupled to the first and second portions. The third portion removably couples to one or more cannulas and places a force upon a base of flared portions of the one or more cannulas when the first and second portions are adhered to the nose. The force maintains a fluid seal of the one or more cannulas within one or more respective nostrils of the nose. The third portion includes a reinforcement. In one configuration, the reinforcement is angled. In another configuration, the reinforcement has a hinge to allow easy adjustment on a nose. In yet another configuration, the reinforcement has stilted holes for cannulas. In yet another configuration, third portion is removably coupleable to the first and second portions.

Before undertaking the DETAILED DESCRIPTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like. The phrase “at least one of,” when used with a list of items, means that different combinations of one or more of the listed items may be used, and only one item in the list may be needed. For example, “at least one of: A, B, and C” includes any of the following combinations: A; B; C; A and B; A and C; B and C; and A and B and C. Definitions for certain words and phrases are provided throughout this patent document, those of ordinary skill in the art should understand that in many if not most instances, such definitions apply to prior, as well as future uses of such defined words and phrases.

### BRIEF DESCRIPTION OF THE DRAWINGS

The various features and advantages of the technology of the present disclosure will be apparent from the following description of particular embodiments of those technologies, as illustrated in the accompanying drawings. It should be noted that the drawings are not drawn to scale; however, the emphasis instead is being placed on illustrating the principles of the technological concepts. Also, in the drawings the like reference characters refer to the same parts throughout the different views. The drawings depict only typical embodiments of the present disclosure and, therefore, are not to be considered limiting in scope.

FIG. 1 shows a full-face mask.

FIG. 2 shows a nose mask.

FIG. 3 shows a cradle.

FIG. 4 shows nasal pillows with straps.

FIG. 5 shows nasal pillows with oral appliance.

FIG. 6 shows a Bleep™ system.

FIG. 7 shows Beevers U.S. Pat. No. 7,331,348 B1.

FIG. 8 shows Kessler U.S. Pat. No. 6,328,038 B1.

FIG. 9 shows Cardoso U.S. Pat. No. 6,669,712 B1.

FIG. 10 shows Kooij U.S. Pat. No. 8,291,906 B2.

FIG. 11 shows Kooij US 2011/0067704 A1.

FIG. 12 shows Barlow US 2012/0138060 A1.

FIG. 13 shows Holtzapple U.S. Pat. No. 10,238,827 B2.

FIG. 14 shows Holtzapple U.S. Pat. No. 10,238,827 B2 with notch and crease.

FIG. 15 show a table comparing the designs of FIGS. 1 through 14.

FIGS. 16A and 16B show improved adhesive strip for securing nasal pillows.

FIG. 17 shows a patient with nasal pillows inserted into the nostrils, which allows for a positive airway pressure (PAP) system to pressurize the breathing passages.

FIG. 18A, 18B, and 18C shows nasal pillows secured with an adhesive strip.

FIG. 19 shows an adhesive strip with multiple slits along the edge of each hole.

FIG. 20 shows an adhesive strip with two slits along the edge of each hole.

FIG. 21 shows an adhesive strip with one slit along the edge of each hole.

FIG. 22 shows components of a nasal pillow mask system.

FIGS. 23A and 23B show an improved adhesive strip with a rigid angle.

FIG. 24A and 24B show an improved adhesive strip with a flexible “hinge.”

FIGS. 25A and 25B show a separated reinforcement with rigid angle.

FIG. 26A and 26B shows a separated reinforcement with flexible “hinge” consisting of flexible adhesive fabric.

FIG. 27A and 27B show a reinforcement with flexible “hinge” consisting of thinned plastic.

FIGS. 28A and 28B shows a reinforcement with rigid angle and connector.

FIGS. 29A and 29B shows a separated reinforcement with connector and flexible “hinge” consisting of flexible adhesive fabric.

FIGS. 30A and 30B show a reinforcement with connector and flexible “hinge” consisting of thinned plastic.

### DETAILED DESCRIPTION

The figures described below, and the various embodiments used to describe the principles of the present disclosure in this patent document are by way of illustration only and should not be construed in any way to limit the scope of the disclosure. Those skilled in the art will understand that the principles of the present disclosure invention may be implemented in any type of suitably arranged device or system. Additionally, the drawings are not necessarily drawn to scale.

### Survey of Existing Technology

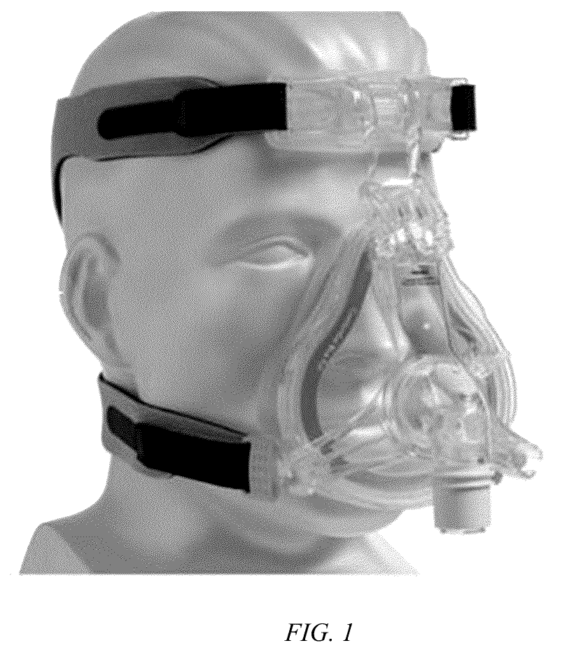

FIG. 1 shows a full-face mask in which pressurized air is supplied through the hose connection to both the nose and mouth. The mask is secured to the face with head straps. Because the shape of each individual's head differs, it is difficult to achieve a secure seal. Furthermore, facial movement easily disrupts the seal.

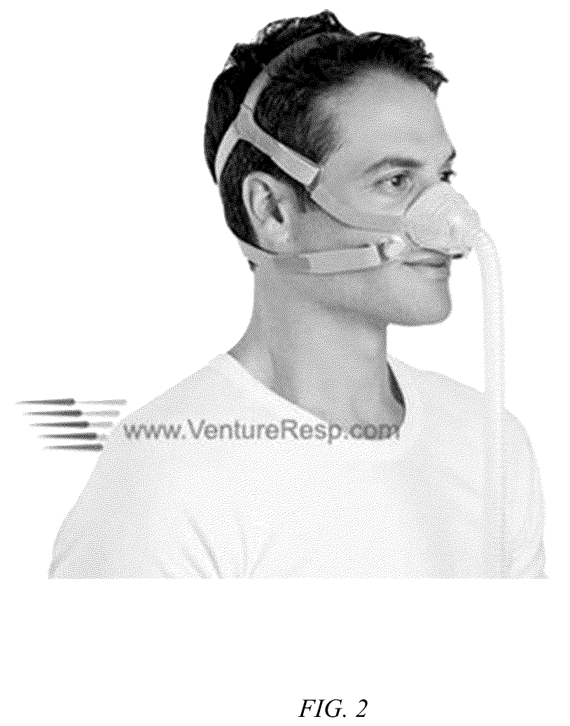

FIG. 2 shows a nose mask in which pressurized air is supplied through the hose connection to the nose. The mask is secured to the nose with head straps. Because the shape of each individual's nose differs, it is difficult to achieve a secure seal. Furthermore, facial movement easily disrupts the seal.

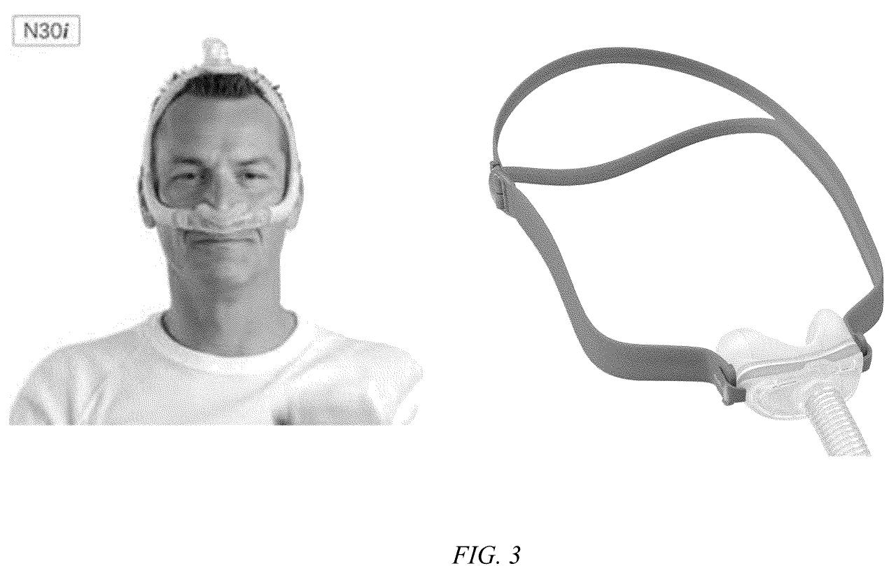

FIG. 3 shows an air-delivery system in which pressurized air is supplied through the hose connection to both nostrils via a cradle. It is secured to the nostrils with head straps. Because the shape of each individual's nostrils differs, it is difficult to achieve a secure seal. Furthermore, facial movement easily disrupts the seal.

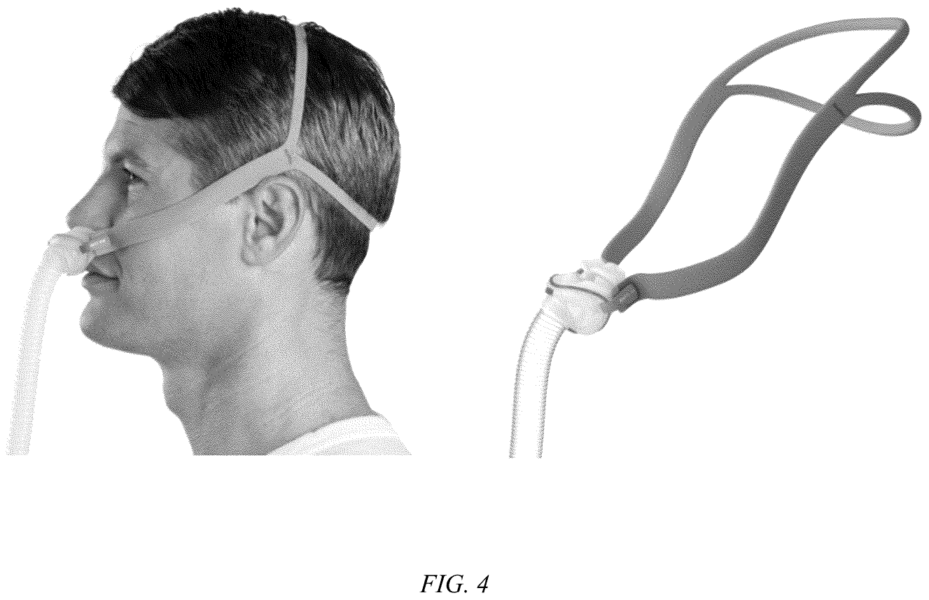

FIG. 4 shows an air-delivery system in which pressurized air is supplied through the hose connection to nasal pillows coupled to each nostril. It is secured to the nostrils with head straps. Although the shape of each individual's nostrils differs, a variety of sizes are available that allow a secure seal; however, facial movement easily disrupts the seal.

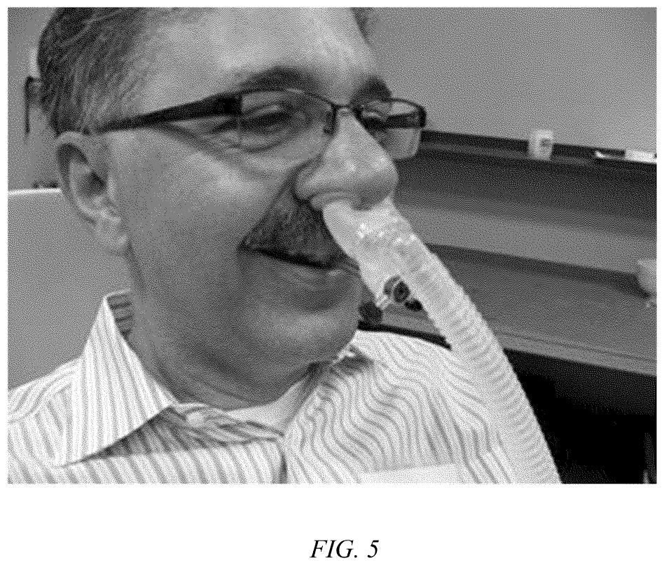

FIG. 5 shows an air-delivery system in which pressurized air is supplied through the hose connection to nasal pillows coupled to each nostril. It is secured to the nostrils with an oral appliance held in place with clenched teeth. Although the shape of each individual's nostrils differs, a variety of sizes are available that allow a secure seal. Although the oral appliance can secure the nasal pillows, the user must clench teeth consistently or lose the seal.

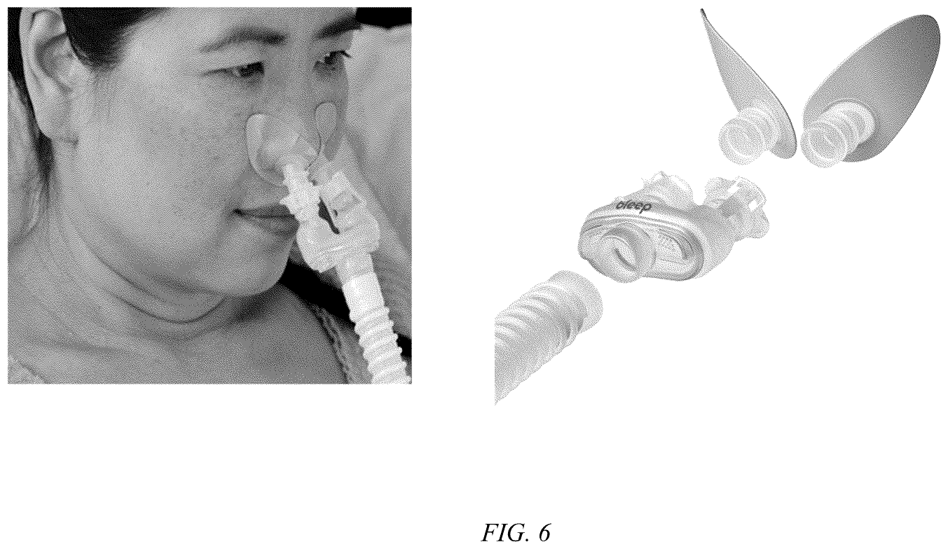

FIG. 6 shows a Bleep™ air-delivery system in which pressurized air is supplied via the hose connection to each nostril through custom ports. The custom ports are joined to adhesive strips that both seal and mechanically secure the device to the nose. The shape of each individual's nostrils differs making it difficult for a single product to seal properly to all noses. Furthermore, the rim of the nostril is very thin, making it difficult to install properly to ensure a good seal.

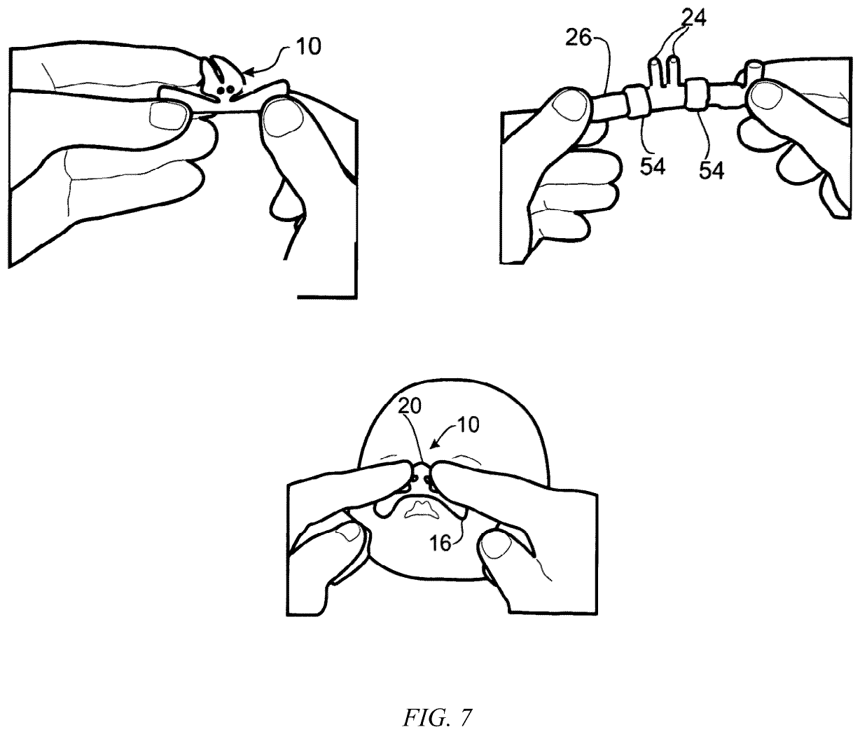

FIG. 7 shows images from the Beevers patent (namely, FIGS. 3C, 4C, and 3E) in which an adhesive strip is secured to the nose thus sealing and covering the nostrils entirely. The adhesive strip has two circular holes through which a cylindrical cannula is placed. The holes seal to the cannula and provide mechanical support. The sealing area between the adhesive strip and the cannula is very small; thus, it is difficult to obtain a good seal. Furthermore, facial movement easily disrupts the mechanical seal thus causing leakage.

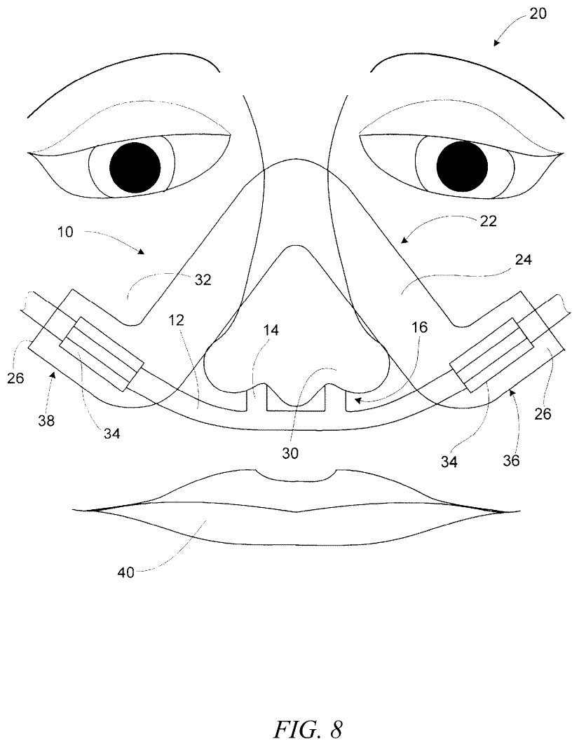

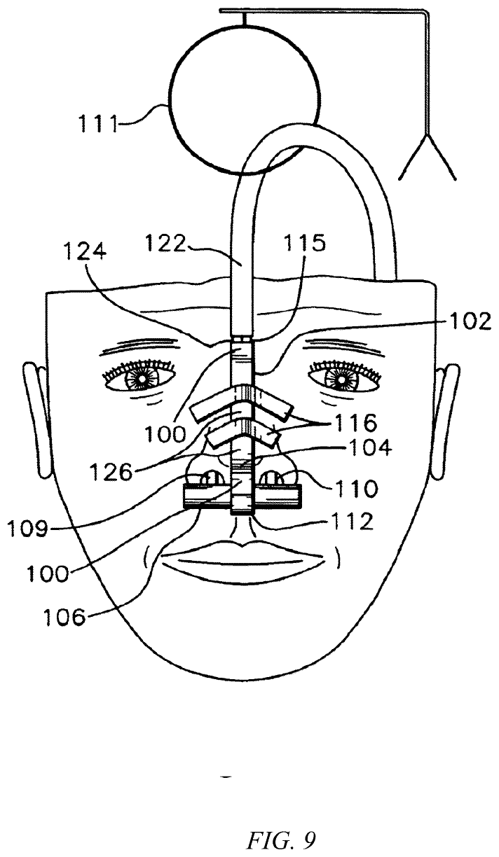

FIGS. 8 and 9 respectively shows image from the Kessler patent (namely, FIG. 2) and Cardoso patent (namely, FIG. 7) that mechanically secures a cylindrical cannula to the nostrils. Because there is no seal, this device is only suitable for delivering oxygen-enriched air at atmospheric pressure and is not able to deliver pressurized air.

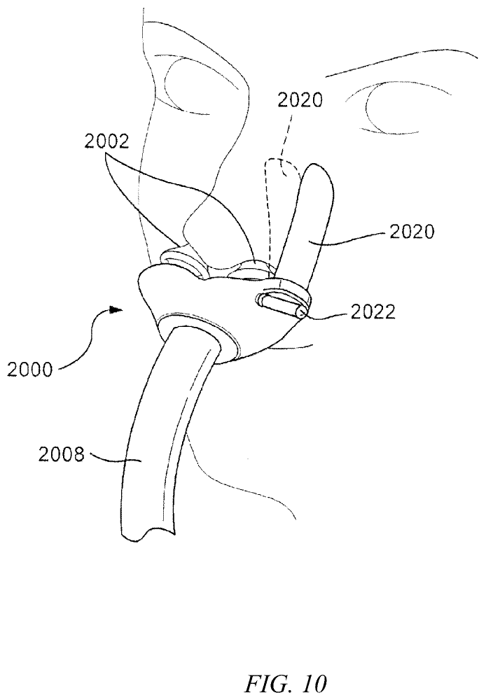

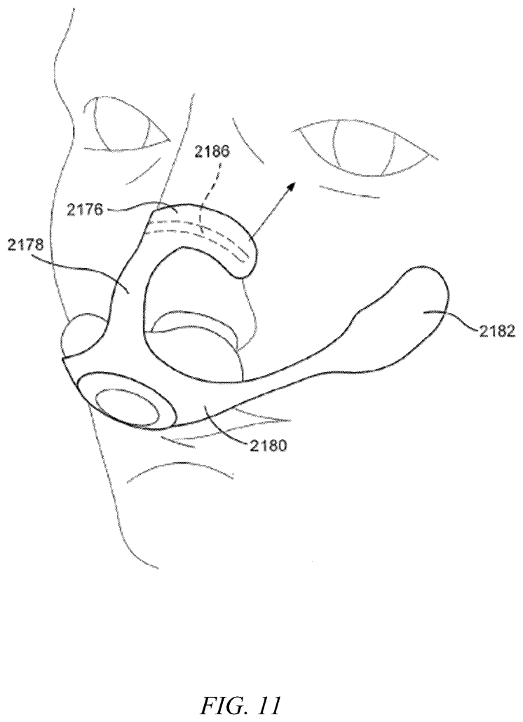

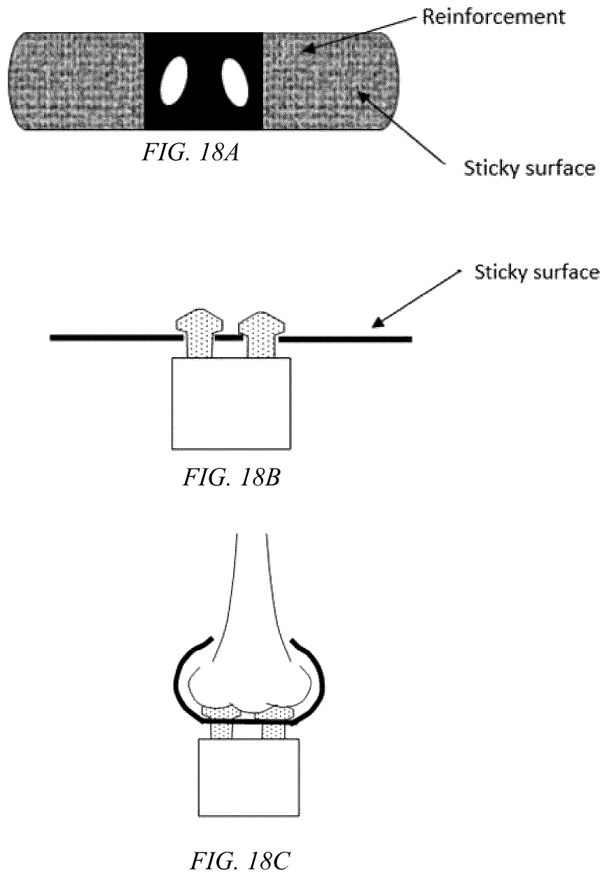

FIGS. 10 and 11 respectively show images from a Kooij patent (namely, FIG. 7) and a Kooji patent application (namely, FIG. 18) in which the air-delivery system is attached to adhesive strips that adhere to the nose. Sealing to the nostrils is accomplished using nasal pillows. The adhesive strips are designed for one-time use and must be disposed; thus, a means of conveniently removing and replacing the adhesive strips must be provided. This design separates the sealing function (nasal pillows) from the mechanical function (adhesive strips), which makes the system robust. The air-delivery system must be designed to accommodate the adhesive strips; thus, this system does not universally accommodate all air-delivery systems that employ nasal pillows. Because the point of attachment is physically distant from the nasal pillows, the moment of force is large so facial movement can easily dislodge the nasal pillows.

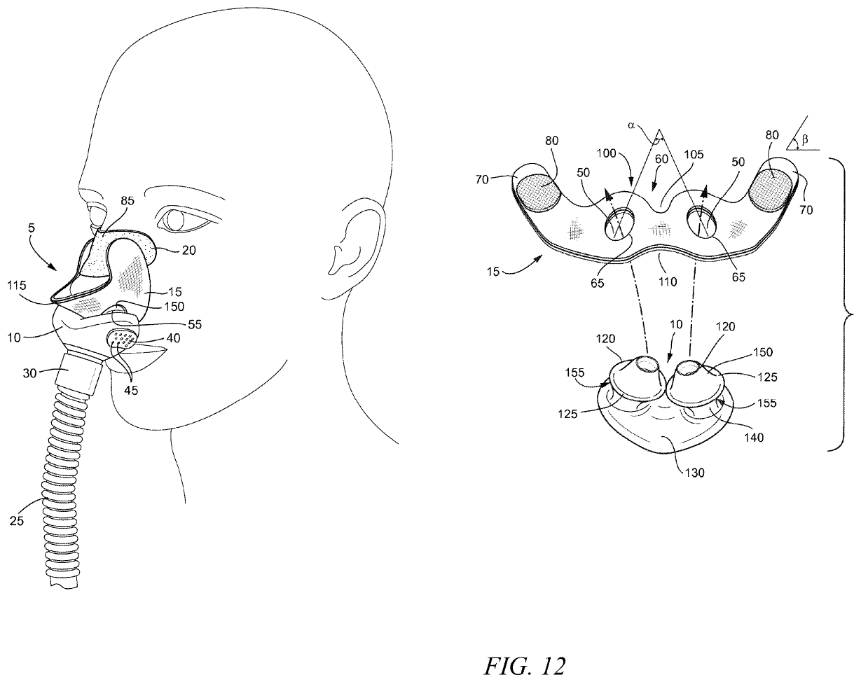

FIG. 12 shows images from the Barlow patent application (namely FIGS. 1 and 6) in which an adhesive strip is attached to the bridge of the nose. The exterior surface of the adhesive strip 20 is covered with one portion of miniature hook-and-loop attachments, commonly marketed as Velcro®. The attachment pads 80 of the small headgear 15 contain the complementary portion of the hook-and-loop attachment. The adhesive strip 20 is designed for one-time use and must be disposed after use. The small headgear 15 has two holes through which the flared ends of the nasal pillows are pushed. This design separates the sealing function (nasal pillows) from the mechanical function (adhesive strips), which makes the system robust. The small headgear 15 can adapt to a wide variety of air-delivery systems, thus this attachment system universally accommodates all air- delivery systems that employ nasal pillows. Because the point of attachment is physically close to the nasal pillows, the moment of force is small; thus, facial movement does not easily dislodge the nasal pillows. On the other hand, because the area of the attachment pads is small, the mechanical bond between small headgear 15 and adhesive strip 20 is weak; thus, facial movement can easily dislodge the nasal pillows.

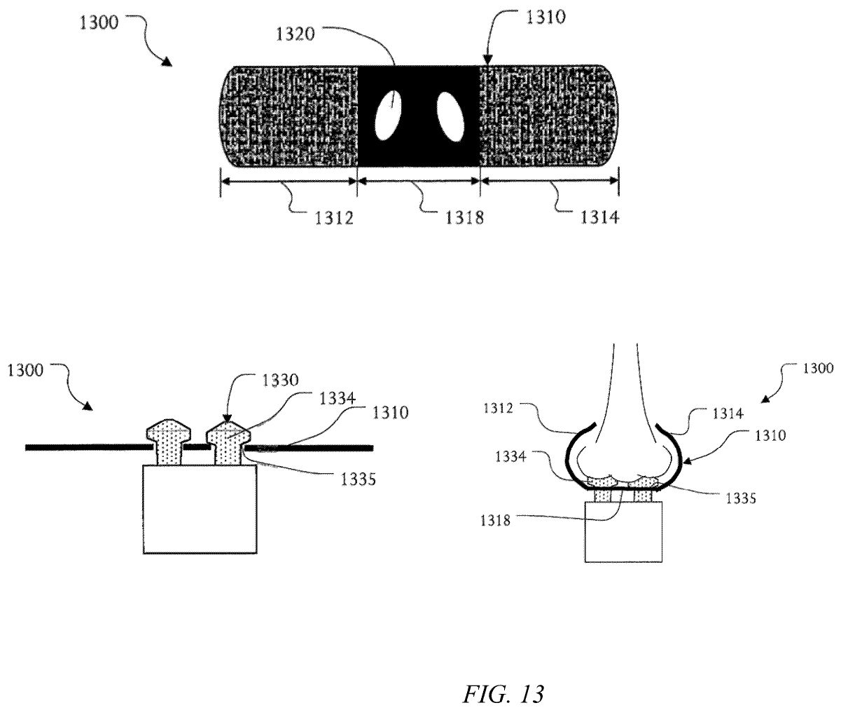

FIG. 13 shows images from the Holtzapple patent (namely, FIGS. 13A, 13B, and 13C) in which an adhesive strip contains two holes surrounded by non-sticky reinforcement material. The flared ends of the nasal pillows are pushed through the holes. The adhesive surface attaches to the wing (alar) of the nose and secures the nasal pillows to the nostrils. This design separates the sealing function (nasal pillows) from the mechanical function (adhesive strips), which makes the system robust. Because the point of attachment is physically close to the nasal pillows, the moment of force is small; thus, facial movement does not easily dislodge the nasal pillows.

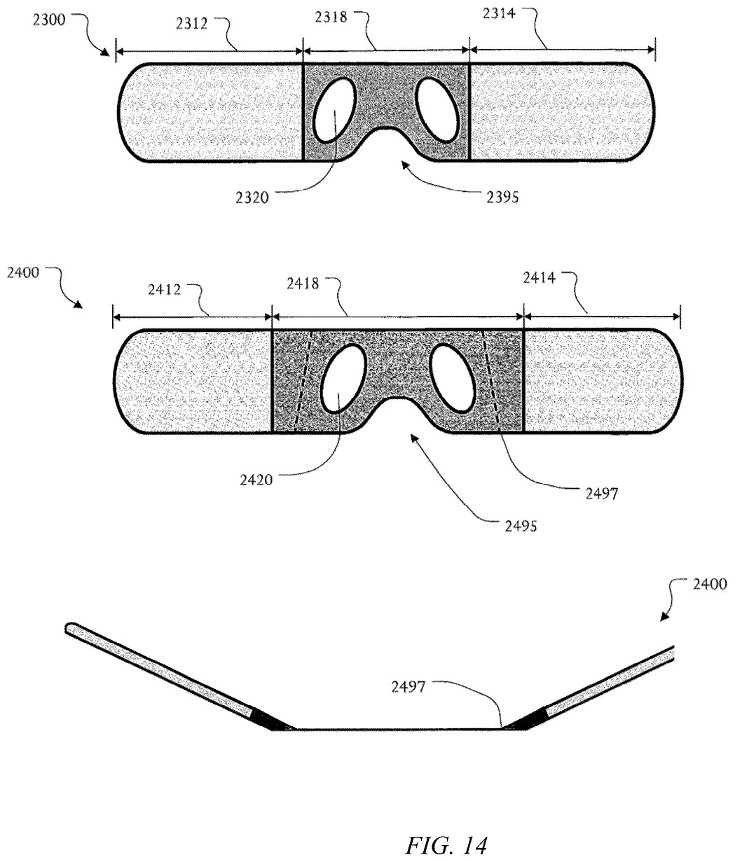

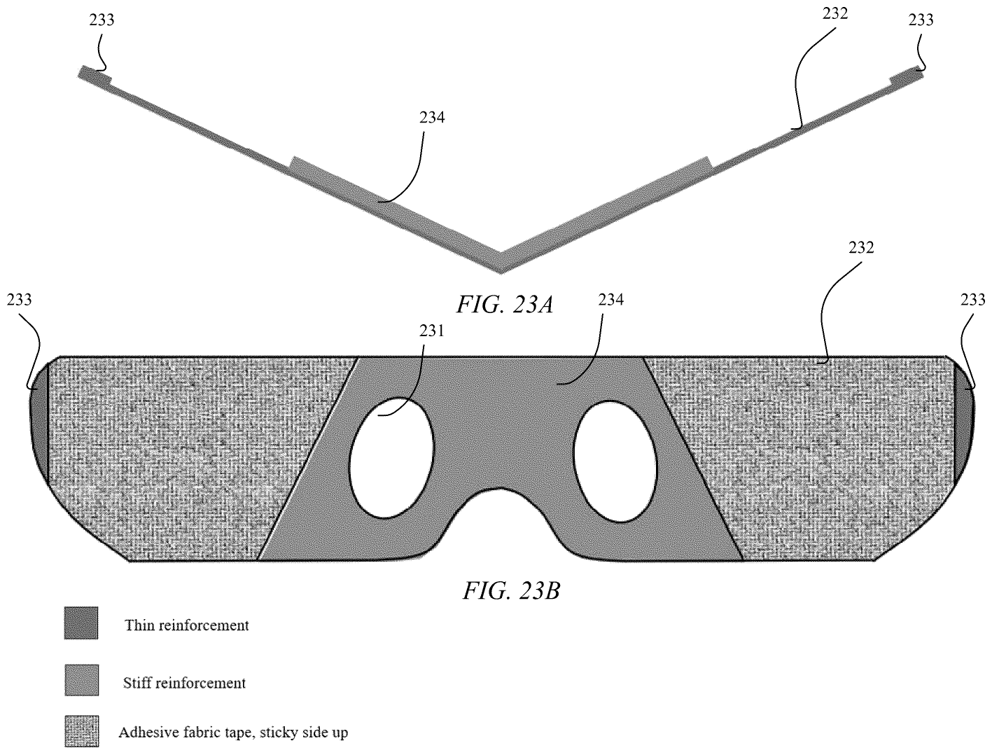

FIG. 14 shows alternative embodiments from the Holtzapple patent (namely FIGS. 23, 24, and 25) in which a notch 2495 accommodates the columella, the fleshy part of the nose that separates the two nostrils. Additionally, a crease 2497 establishes a fold line that assists in the orientation of the adhesive strip onto the nose.

### Comparison of Existing Technologies

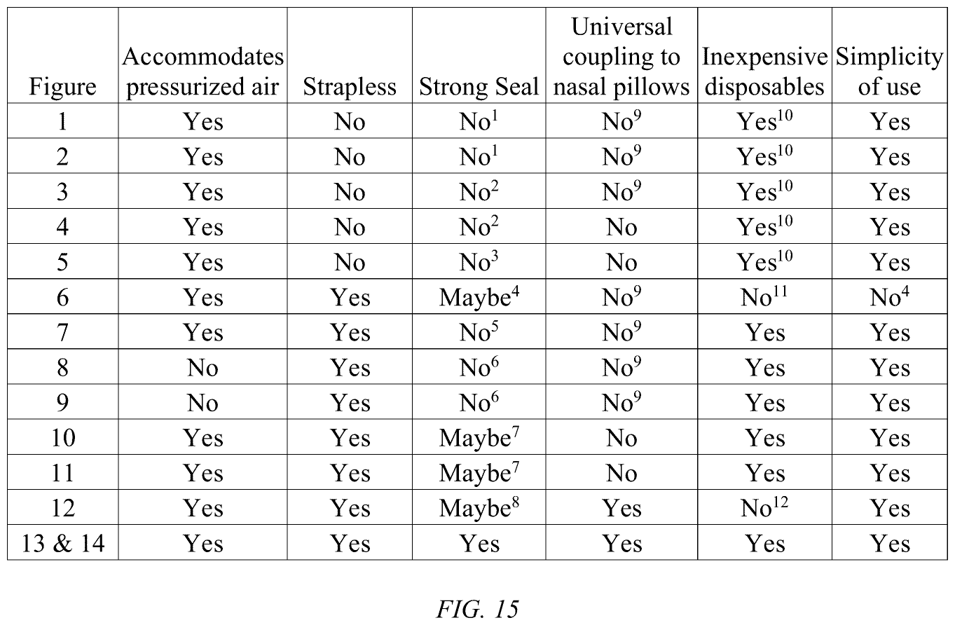

FIG. 15 is a table that compares the existing technologies described above with reference to FIGS. 1 through 14. Assessments were made on the following bases:
Accommodates pressurized air—Systems that create a seal and can accommodate pressurized air are more versatile.
Strapless—Straps are undesirable because they create a claustrophobic effect, damage hair, and created indentations on the face.
Strong seal—Explanations for poor seals are given in the footnotes.
Universal coupling to nasal pillows—Technologies that provide universal coupling to nasal pillows made by multiple vendors provides users with maximum choice and flexibility.
Inexpensive disposables—Many applications (e.g., sleep apnea) require daily use of disposables, so lowering the cost is important for widespread acceptance.
Simplicity of use—Technologies that are difficult to employ will not gain widespread acceptance.

The following correspond to the particular super-script notations for each indicated item.
1. Difficult to seal against highly variable facial and nose structures.
2. Easily dislodged upon facial movement
3. Loses seal when slackens.
4. Small sealing around perimeter of nostril makes it difficult to install properly.
5. Small sealing area between hole in adhesive strip and cylindrical cannula.
6. Has no sealing surface.
7. Large movement of force cause easy dislodgement upon facial movement.
8. Small attachment surface easily dislodge upon facial movement.
9. Does not employ nasal pillow.
10. No disposables required.
11. Ports are expensive and difficult to interfere with adhesive strip
12. Hook-and-loop attachment is expensive.

Based on the analysis provided in Table of FIG. 15, the Holtzapple technology has the most desirable features.

### Improvements on Existing Technology

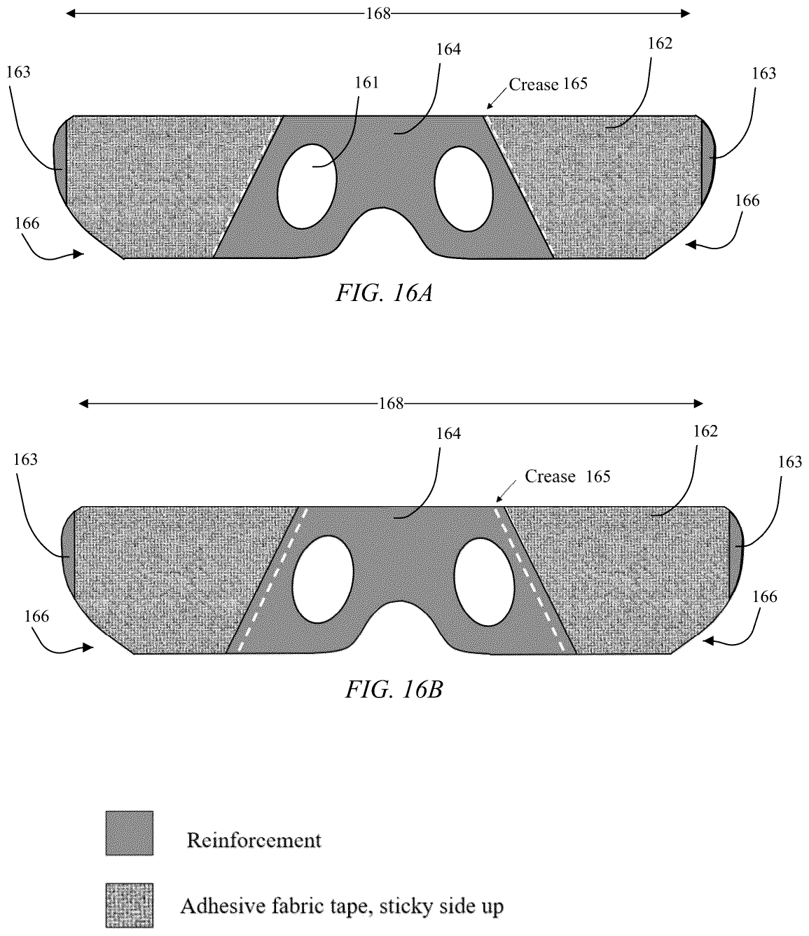

FIGS. 16A and 16B shows embodiment of the Holtzapple technology with improved features. Preferably, the adhesive strip 162 is comprised of a length of medical-grade adhesive fabric tape coated with medical-grade pressure-sensitive acrylic adhesive. Although such a material is described in this configuration, the adhesive strip 162 may be made of other materials in other configurations.

Layered on the adhesive fabric tape is a non-sticky polymer reinforcement 164 such as polyethylene terephthalate (PET) or other suitable material. To maximize the adhesive area of the adhesive strip, the edges of the polymer reinforcement 164 are at an oblique angle relative to the long axis 168; although a particular angle is shown, other may be used in other configurations. Holes 161 extend through the reinforcement 164 and adhesive strip 162.

The length along the long axis 168 is extended to touch the bridge of the nose. To avoid touching the eyes, material is removed from the bottom left and bottom right portions of the adhesive area (as indicated by arrow 166). While one particular shape for removal of materials is shown, other appropriate removal of materials may also be utilized.

The angle of the crease 165 and the angle of the edge of the reinforcement 164 may be essentially identical; thus, the stiffness provided by the reinforcement helps define the folding edge. Preferably, if the crease 165 is formed by embossing the adhesive fabric tape, the crease 165 is located on the edge of the reinforcement, which allows the stiffness of the reinforcement to help defined the fold line (FIG. 16A). Preferably, if the crease is formed by perforating or cutting the adhesive fabric tape, the crease 165 is located within the reinforcement, which provides additional mechanical strength to ensure the crease 165 does not fail (FIG. 16B).

Stickiness is removed from the tips 163, which allows for easy peel-away removal.

As a recapitulation of features described in FIGS. 16A and 16B, an adhesive strip affixes nasal pillows to the nostrils thus providing a robust seal for breathing-assist devices that supply pressurized air to the lungs. The adhesive strip is comprised of medical-grade adhesive fabric layered with non-sticky polymer reinforcement in the center and the tips. The nasal pillows are supported by two holes located in the center of the adhesive strip. The shape of the adhesive strip is optimized to maximize mechanical contact with the wings (alar) and bridge of the nose while simultaneously avoiding contact with the eyes. A crease is oriented with the polymer reinforcement to assist proper alignment during installation. The tips of the adhesive strip are not sticky, thus facilitating easy removal.

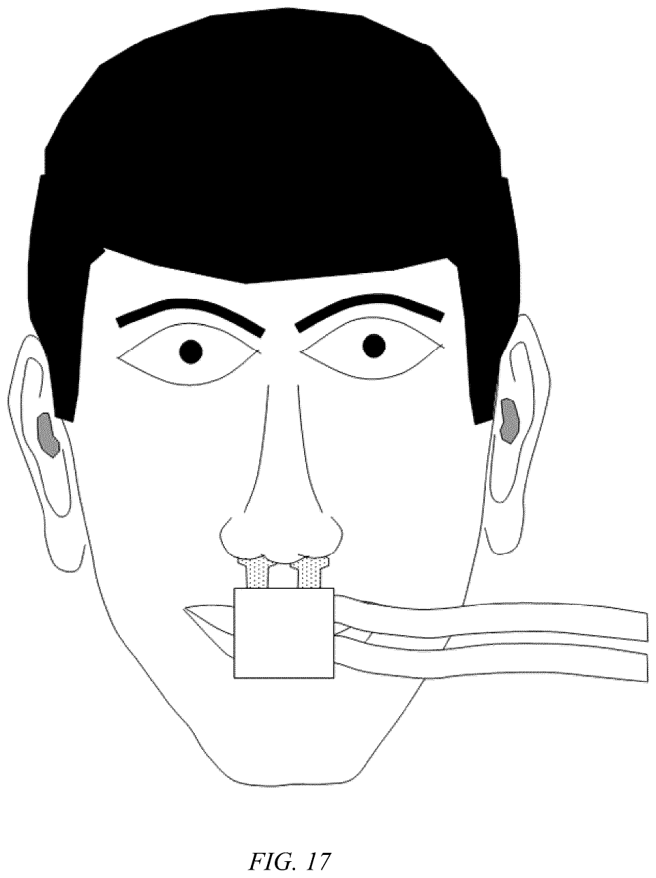

FIGS. 17 and 18A, 18B, and 18C provide context for additional embodiments of the disclosure.

FIG. 17 shows a patient with nasal pillows inserted into the nostrils, which allows a positive airway pressure (PAP) system to pressurize the breathing passages and thus ensure there are no obstructions that can cause sleep apnea and/or snoring. In this illustration, the nasal pillows are not mechanically secured to the nostril, so leaks will occur even if the nasal pillows are secured with a strap.

Nasal pillows with a variety of features are made by multiple manufacturers. To ensure the best results, it is desirable for patients to select the type of nasal pillow that works best for them. Ideally, a system that addresses leaks should be “universal,” thus allowing the patient complete freedom to select the nasal pillow that best addresses their needs.

FIG. 18A shows an adhesive strip with two holes. FIG. 18B shows a side view in which the nasal pillows have been inserted through the holes of the adhesive strip. In some cases, particularly when the patient has arthritis or other dexterity-limiting issues, it can be difficult to insert the nasal pillows through the holes. Furthermore, the difficulty is exacerbated when using gel-type nasal pillows, which are very thick and inflexible. FIG. 18C shows the nasal pillows mechanically secured to the nostrils using an adhesive strip with two holes.

To overcome potential difficulties associated with inserting the nasal pillows into the holes of the adhesive strips, the embodiments herein disclose slits that can be cut along the edge of the hole to facilitate insertion.

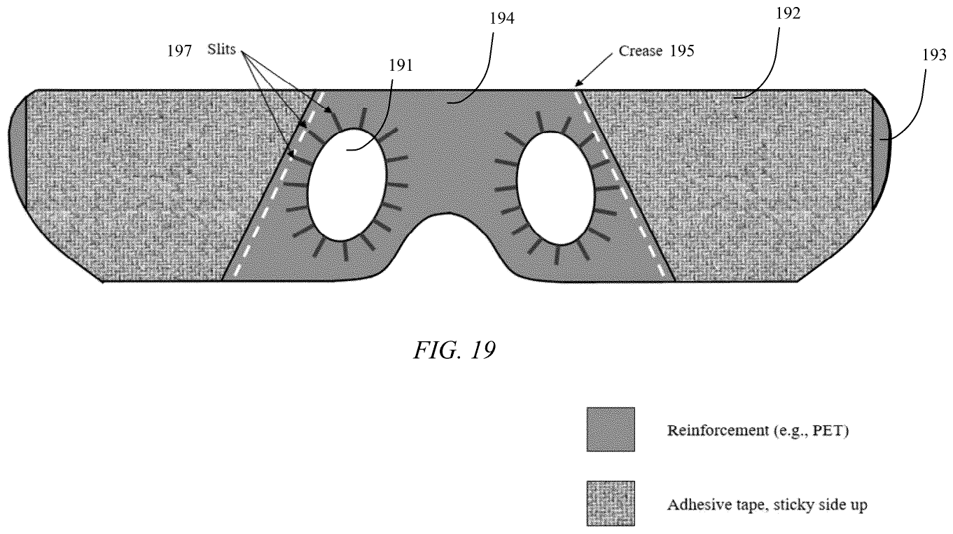

FIG. 19 has features similar to FIGS. 16A and 16B, including an adhesive strip 192, a reinforcement portion 194 at an angle with respect to the adhesive strip 192, an angled crease 195, tips 193, and holes 191. However, FIG. 19 also has slits 197 along the edge of each hole 191. The slits 197 allow maneuvering of the pillows through the holes 191 by effectively increasing the area available for the pillows to pass through during such maneuvering. Although the slits 197 of FIG. 19 have been shown in the context of the configuration of FIGS. 16A and 16B, the slits may be used in other configurations—including the configurations of FIGS. 18A, 18B, and 18C.

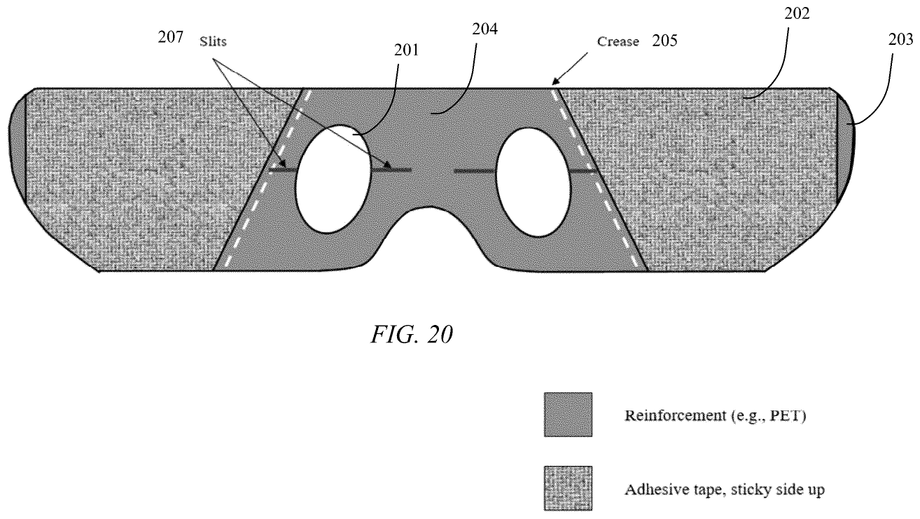

FIG. 20—like FIG. 19—has features similar to FIGS. 16A and 16B, including an adhesive strip 202, a reinforcement portion 2024 at an angle with respect to the adhesive strip 202, an angled crease 205, tips 203, and holes 201. FIG. 20 shows two slits 207 along the edge of each hole 201. The slits 207 allow maneuvering of the pillows through the holes 201 by effectively increasing the area available for the pillows to pass through during such maneuvering. Although the slits 207 of FIG. 20 have been shown in the context of the configuration of FIGS. 16A and 16B, the slits may be used in other configurations—including the configurations of FIGS. 18A, 18B, and 18C.

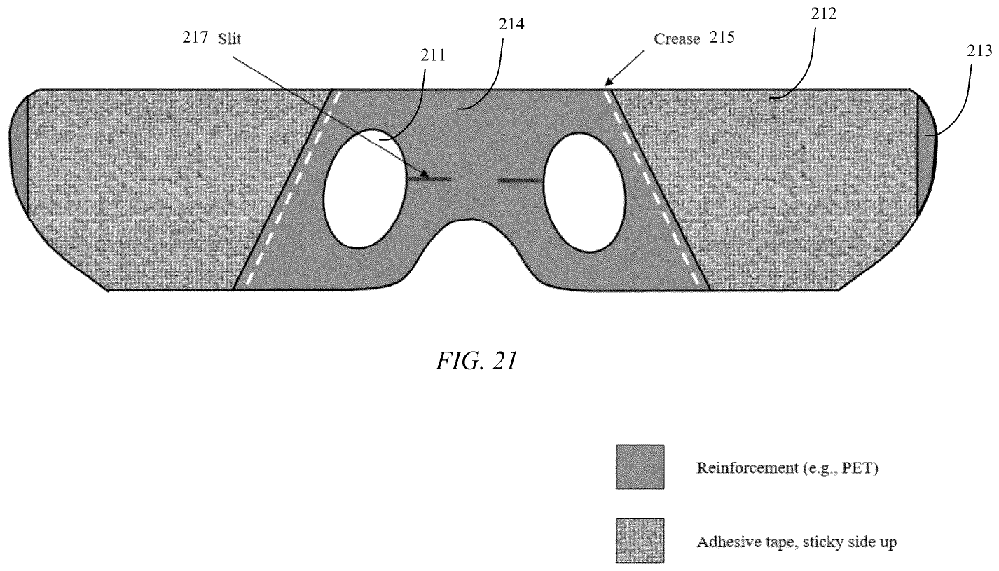

FIG. 21—like FIGS. 19 and 20—has features similar to FIGS. 16A and 16B, including an adhesive strip 212, a reinforcement portion 214 at an angle with respect to the adhesive strip 212, an angled crease 215, tips 213, and holes 211. FIG. 21 shows a single slit 217 along the edge of each hole 211.

With reference to FIGS. 19, 20, and 21, the reinforcement can be made from a flexible material, such as polyethylene terephthalate (PET). The reinforcement provides “springiness” that allows the respective slit or slits to open when inserting the nasal pillow, but also provides “stiffness” that helps mechanically secure the nasal pillows once inserted into the nose.

As a recapitulation of the feature of FIGS. 18, 19, and 20, adhesive strips that secure nasal pillows to the nostrils ensure a leak-tight seal for positive airway pressure (PAP) systems. Inserting the nasal pillows into the holes of the adhesive strip can be difficult, particularly for patients with arthritis or other dexterity-limiting issues. Incorporating slits along the edges of the holes make it easier to insert the nasal pillows through the holes.

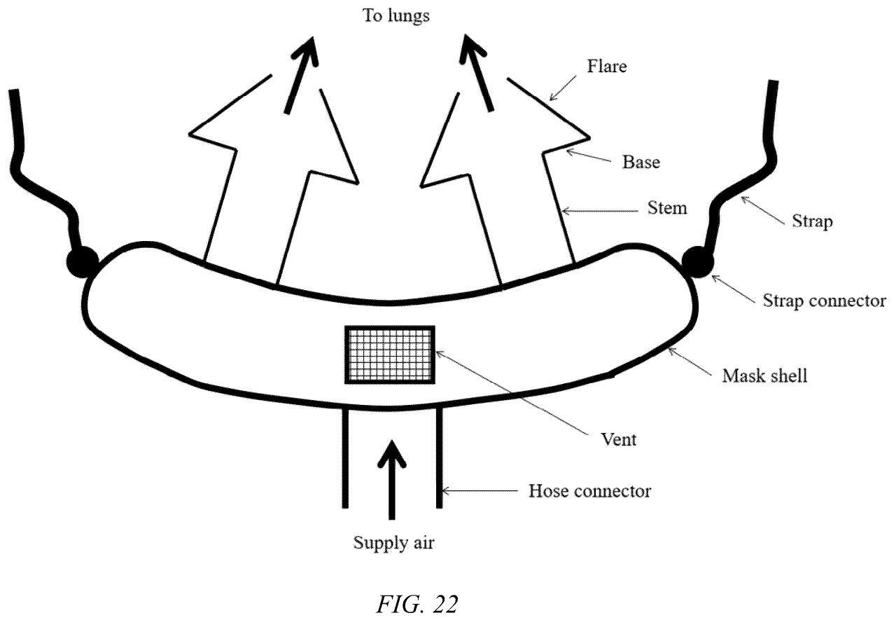

FIG. 22 provides context for additional embodiments of the disclosure. In particular, FIG. 22 shows the components of a typical nasal pillow mask. The flare, base, and stem are made from a flexible material, such as silicone rubber. Typically, the mask shell and hose connector are made from rigid plastic.

The flares are inserted into the patient's nostrils. As air pressure is supplied from the blower (not shown), each flare expands and seals against the nostrils. Upon inhalation, pressurized air is supplied to the lungs. Upon exhalation, exhaust air exits the vent and enters the ambient atmosphere.

Typically, the mask shell connects to head straps that secure the nasal pillows into the nostrils. Because straps move easily and the mask shell is distant from the nostrils, movements of the head easily dislodge the flares from the nostrils causing leaks. Unfortunately, the leaks generate noise that can awaken the patient. Furthermore, leaks can reduce the pressure at the mask, which reduces efficacy.

To properly seal a nasal pillow to the nose, it is necessary to apply a securing force directly to the base of the nasal pillow. This can be accomplished by inserting the nasal pillows through holes in an adhesive strip that attaches to the exterior of the nose. The use of an adhesive strip can eliminate the need for head straps, which improves patient comfort and eliminates damage to hair.

The holes in the adhesive strip are surrounded by non-sticky reinforcement. The reinforcement can be flexible, like the adhesive strip, or it can be stiff. For example, stiff reinforcement can be achieved with polyethylene terephthalate (PET) with a thickness of 0.012 inches. A stiff reinforcement allows the nasal pillows to seal against higher pressures; however, stiffness can make it difficult to accommodate natural variations in patients' nose dimensions.

FIGS. 23A and 23B shows an example of an adhesive strip that contains a stiff reinforcement 234 surrounding the holes 231 in the adhesive strip 232. FIG. 23A shows a cross-sectional view and FIG. 23B shows a top view. Tips 233 may have a thin reinforcement as compared to the reinforcement 234. To accommodate the shape of the nostrils, the stiff reinforcement 234 is divided into two sections joined by a rigid angle. The rigid angle introduces two problems: (1) it is expensive to manufacture and (2) the angle may not match the angle of the patient's nose and thereby prevent a good seal.

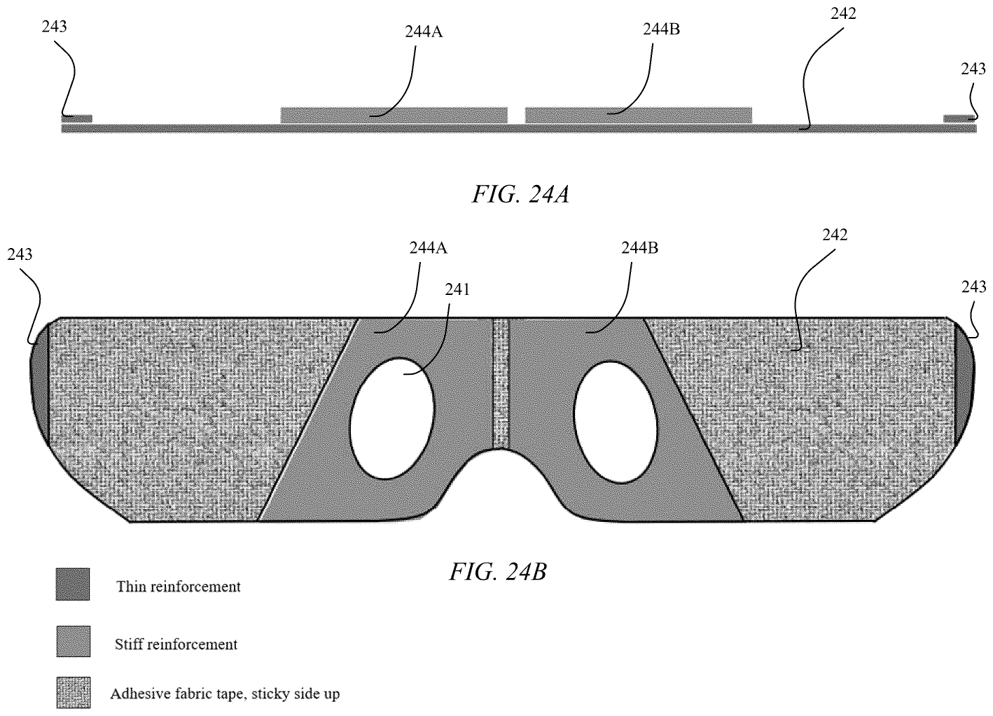

FIGS. 24A and 24B shows an embodiment that addresses the previously mentioned problems with reference to FIGS. 23A and 23B. It includes thin reinforcement tips 243, adhesive strip 242, and holes 241. However, the stiff reinforcement is separated into two sections, respectively labeled 244A and 244B. A sufficient gap (about 0.020 inches) is placed between the two sections such that the adhesive fabric 242 tape serves as a “hinge.” This embodiment allows the stiff reinforcement 244A, 244B to be flat and the hinge easily adapts to the angle of the patient's nostrils. Although a specific gap distance is referenced, a gap of more or less than about 0.020 inches may be used in other configurations. Also, while shown as connected, in particular configurations, a user may be allowed to place the reinforcement sections on to the adhesive fabric 242. Accordingly, for example, a user that has already acquired adhesive strips may retrofit such strips with the reinforcement sections. Alternatively, the configuration of FIGS. 24A and 24B may be provided to a user as a whole. Yet, further, a user may be provided both the adhesive strips 242 and the reinforcement sections to connect on-demand. Such concepts may be also be used with reference to other figures described below.

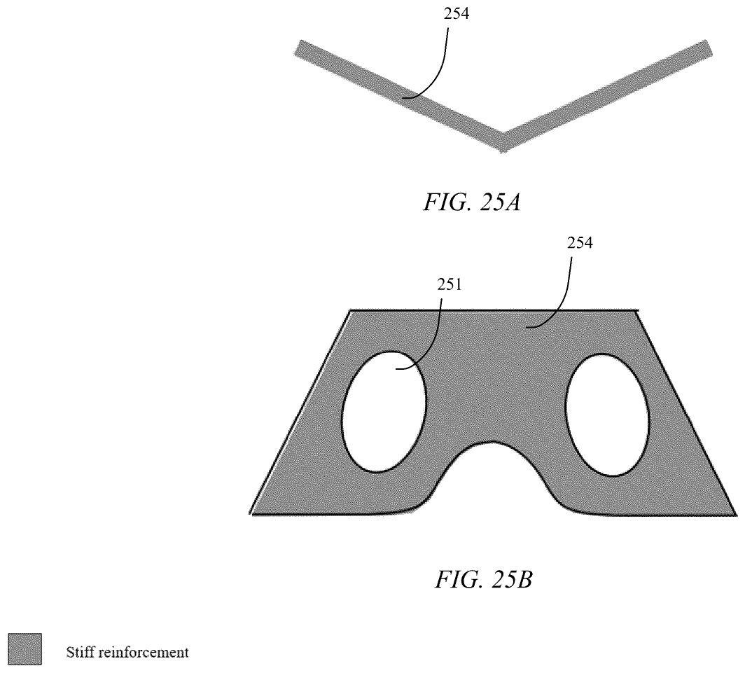

FIGS. 25A and 25B show an embodiment that consists only of the stiff reinforcement 254, which is separated from the adhesive strip. In this embodiment, the nasal pillows would be inserted first through the holes in a flexible adhesive strip (not shown). Then, the nasal pillows would be inserted through the holes 251 in stiff reinforcement 254 shown in FIGS. 25A and 25B. In this embodiment, two layers are required. In this configuration, the layer closest to the base of the nasal pillow is stiff. It is backed with a layer consisting of a flexible adhesive strip that secures the entire assembly to the nose.

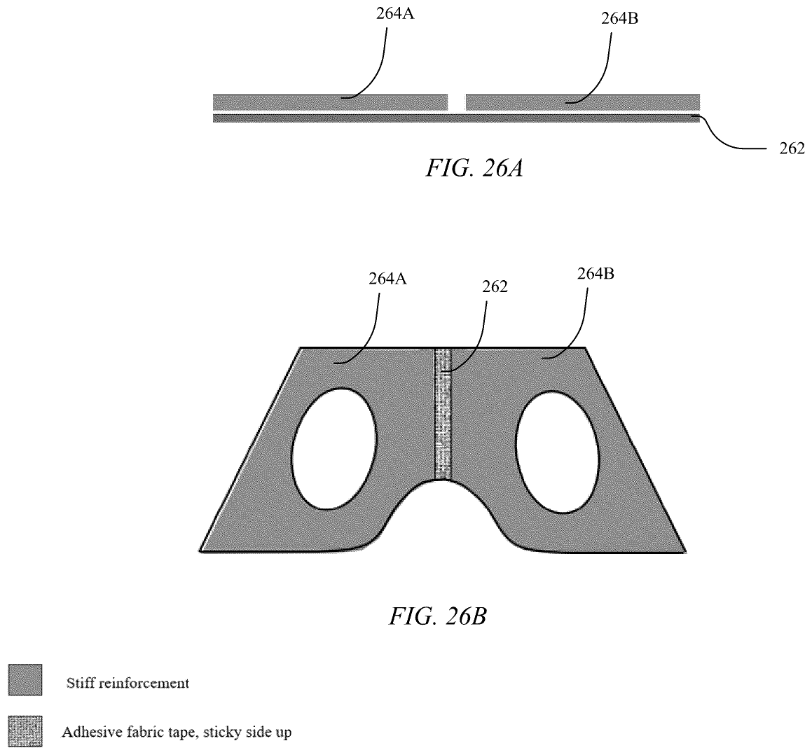

FIGS. 26A 26B, 27A, and 27B shows an embodiment similar to that shown in FIGS. 25A and 25B except that it employs a flexible “hinge.” As shown in FIGS. 26A and 26B, the “hinge” is created by securing adhesive flexible tape 262 to the underside. The reinforcement has two portions, respectively labeled 264A and 264B.

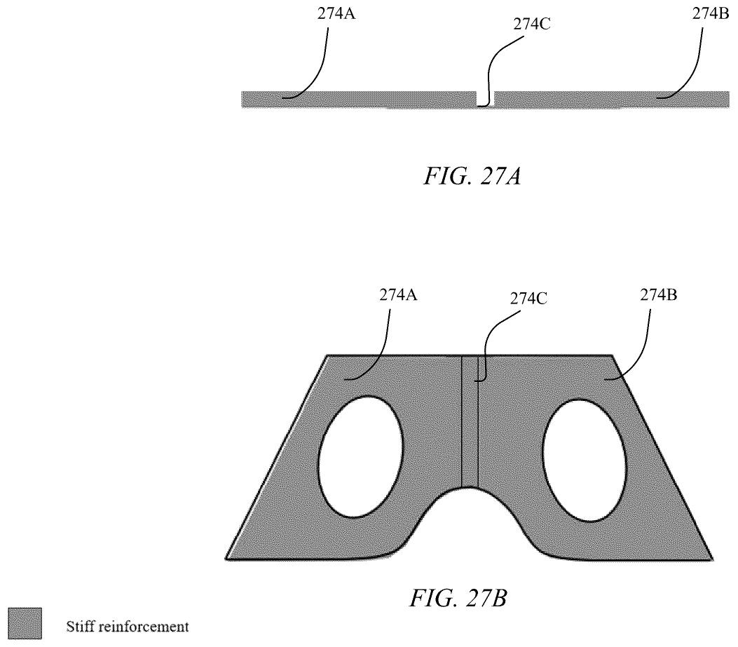

As shown in FIG. 27A and 27B, the “hinge” is created by thinning the middle section 274C of the stiff reinforcement. For example, if the stiff reinforcement is manufactured from a thermoplastic, the thin section 274C can be created by pressing a warm metal bar against the plastic. The thin section 274C serves as a hinge for portions 274A, 274B.

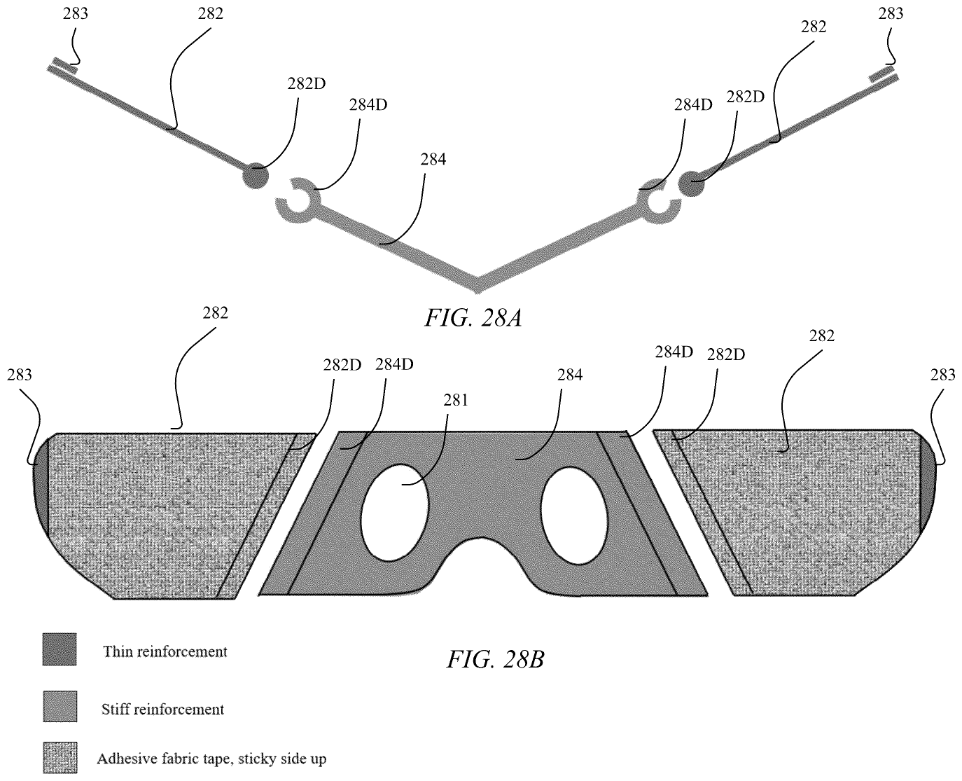

FIGS. 28A and 28B show an embodiment that consists of the stiff reinforcement 284, which is separated from the adhesive strip 282. In this embodiment, the nasal pillows would be inserted through the holes 281 of the stiff reinforcement 284 shown in FIG. 28B. The edges 284D of the stiff reinforcement 284 include attachment means for attaching adhesive tape to the edges. In this embodiment, the edges 284D of the stiff reinforcement contain passageways into which the flared edges 282D of the adhesive tape 282 are inserted. Other example means for connecting the adhesive tape to the stiff reinforcement include hook-and-loop, buttons, snaps, and Velcro®. One of ordinary skill in the art will recognize further means for connecting the adhesive tape to the stiff reinforcement. Thin reinforcement tips 283 are also shown.

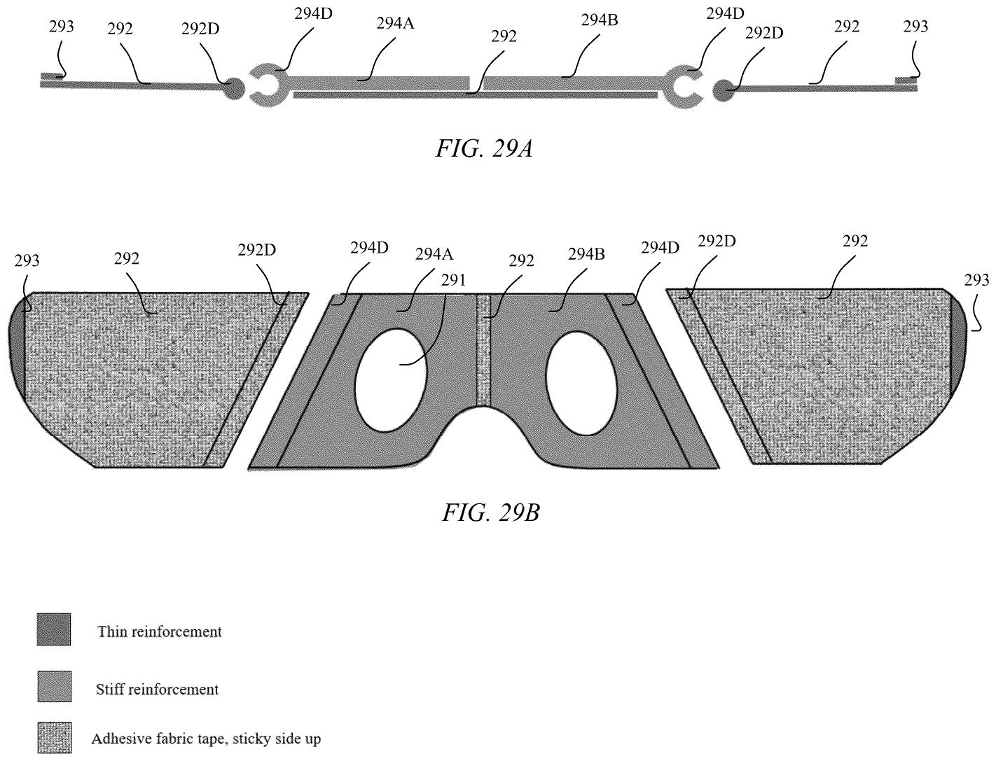

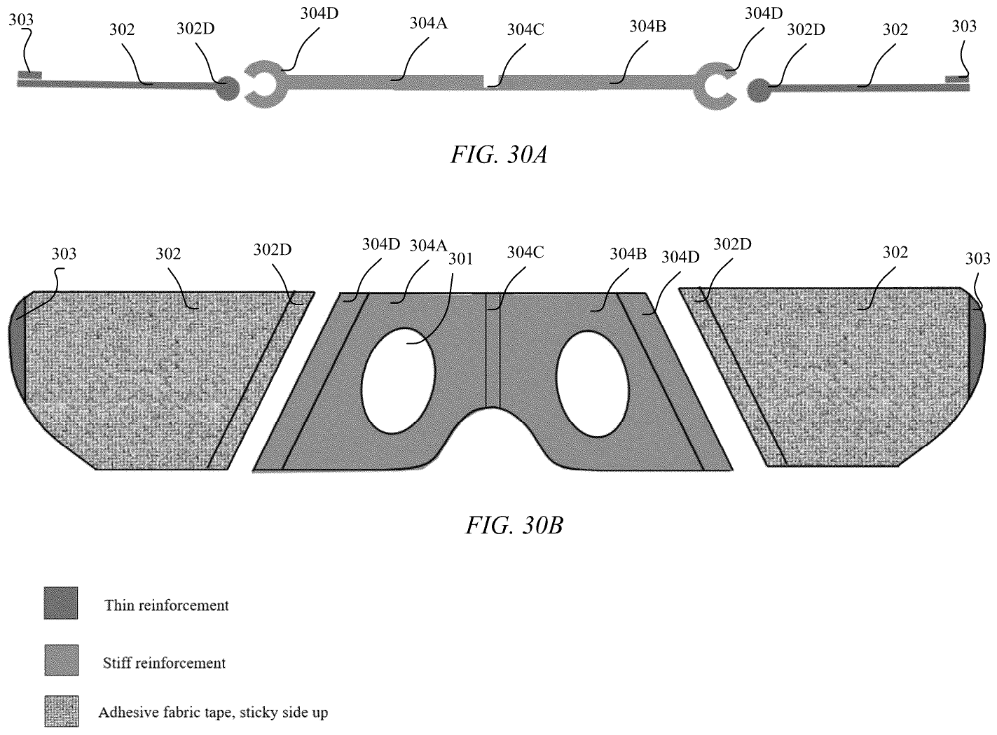

FIGS. 29A, 29B, 30A, and 20B show an embodiment similar to that shown in FIGS. 28A and 28B, except that they employ a flexible “hinge.” The flexible hinge is the adhesive tape 292 in FIGS. 29B whereas the flexible hinge in FIG. 30B is a thinned section 304C of the reinforcement.

In these embodiments, the nasal pillows would respective be inserted through the holes 291, 301 of the stiff reinforcement sections 294A/294B, 304A/304B shown in FIGS. 29B and 30B. The respective edges 294D, 304D of the stiff reinforcement sections 294A/294B, 304A/304B include attachment means for attaching adhesive tape to the edges. In these embodiments, the edges 294D, 304D of the stiff reinforcement sections 294A/294B, 304A/304B contain passageways into which the flared edges 292D, 302D of the adhesive tape 292, 302 are inserted. Other example means for connecting the adhesive tape to the stiff reinforcement include hook-and-loop, buttons, snaps, and Velcro®. One of ordinary skill in the art will recognize further means for connecting the adhesive tape to the stiff reinforcement. Thin reinforcement tips 293, 303 are also shown

Although this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of this disclosure,

What is claimed is:

1. A universal sealing apparatus comprising:
a tape or bandage that comprises:
a first portion configured to removably adhere directly or indirectly to a first outer side of a nose;
a second portion configured to removably adhere directly or indirectly to a second outer side of the nose; and
a third portion between and coupling the first and second portions, the third portion configured to:
removably receive multiple cannulas of a pressurized air-delivery system that are configured to be inserted through the third portion of the universal sealing apparatus and into nostrils of the nose, the cannulas having flared portions, and
place a force upon bases of the flared portions of the cannulas when the first and second portions are adhered to the nose to maintain the flared portions of the cannulas within the nostrils of the nose and to maintain fluid seals of the cannulas with the nose;
wherein the universal sealing apparatus has an elongated shape having (i) first and second longer sides extending along a longer dimension of the universal sealing apparatus and (ii) first and second shorter sides extending along a shorter dimension of the universal sealing apparatus;
wherein the third portion comprises multiple holes between the first and second portions, each hole configured to receive and allow passage of one of the cannulas completely through the third portion;
wherein the third portion also comprises a reinforcement located at least around the holes; and
wherein the reinforcement has a shape with edges that have opposite oblique angles with respect to at least the first longer side of the universal sealing apparatus at opposite ends of the third portion.

2. The apparatus of claim 1, wherein the tape or bandages further comprises multiple creases adjacent the edges of the third portion, and wherein the creases providing straight folding lines for the universal sealing apparatus.

3. The apparatus of claim 2, wherein creases are positioned within the reinforcement.

4. The apparatus of claim 2, wherein creases are positioned outside the reinforcement.

5. The apparatus of claim 2, wherein creases have the same oblique angles as the shape of the reinforcement.

6. The apparatus of claim 1, further comprising:
reinforced tips on the outer edges of the first and second portion.

7. The apparatus of claim 1, wherein the first and second portions have portions removed along a side of the longer dimension, the removed portions configured to avoid a touching of the eyes.

8. The apparatus of claim 1, wherein the third portion further comprises a notch configured to accommodate a columella of the nose.

9. The apparatus of claim 1, further comprising:
at least one slit in the reinforcement for each of the holes, each of the at least one slit for each of the holes configure to ease a maneuverability of the cannulas when inserted through the holes.

10. The apparatus of claim 1, wherein the first, second, and third portions are continuous.

11. The apparatus of claim 1, wherein the reinforcement is carried by the tape or bandage.

12. A universal sealing apparatus comprising:
a tape or bandage that comprises:
a first portion configured to removably adhere directly or indirectly to a first outer side of a nose;
a second portion configured to removably adhere directly or indirectly to a second outer side of the nose; and
a third portion between and coupling the first and second portions, the third portion configured to:
removably receive multiple cannulas of a pressurized air-delivery system that are configured to be inserted through the third portion of the universal sealing apparatus and into nostrils of the nose, the cannulas having flared portions, and
place a force upon bases of the flared portions of the cannulas when the first and second portions are adhered to the nose to maintain the flared portions of the cannulas within the nostrils of the nose and to maintain fluid seals of the cannulas with the nose;
wherein the universal sealing apparatus has an elongated shape having (i) first and second longer sides extending along a longer dimension of the universal sealing apparatus and (ii) first and second shorter sides extending along a shorter dimension of the universal sealing apparatus;
wherein the third portion comprises multiple holes between the first and second portions, each hole configured to receive and allow passage of one of the cannulas completely through the third portion;
wherein the third portion also comprises a reinforcement located at least around the holes, the reinforcement having a first and second portion; and
wherein the tape or bandage has a hinge between the first and second portions of the reinforcement.

13. The apparatus of claim 12, wherein the hinge comprises fabric separating the first and second portions of the reinforcement.

14. The apparatus of claim 12, wherein
the fabric is the tape or bandage, and
the reinforcement is carried by the tape or bandage.

15. The apparatus of claim 12, wherein the third portion further comprises a notch configured to accommodate a columella of the nose.

16. The apparatus of claim 12, wherein the first, second, and third portions are continuous.

17. The apparatus of claim 12, wherein the reinforcement has a shape with edges that have opposite oblique angles with respect to at least the first longer side of the universal sealing apparatus at opposite ends of the third portion.

18. The apparatus of claim 17, wherein the tape or bandages further comprises multiple creases adjacent the edges of the third portion, and wherein the creases providing straight folding lines for the universal sealing apparatus.

19. The apparatus of claim 18, wherein creases are positioned within the reinforcement.

20. The apparatus of claim 18, wherein creases are positioned outside the reinforcement.

21. The apparatus of claim 18, wherein creases have the same oblique angles as the shape of the reinforcement.

22. The apparatus of claim 12, further comprising:
reinforced tips on the outer edges of the first and second portion.

23. The apparatus of claim 12, wherein the first and second portions have portions removed along a side of the longer dimension, the removed portions configured to avoid a touching of the eyes.

24. The apparatus of claim 12, wherein the third portion further comprises a notch configured to accommodate a columella of the nose.

25. The apparatus of claim 12, wherein
the hinge is made of the same materials as the first and second portion of the reinforcement,
the hinge comprises a thinner layer of the material as compared to the first and second portion of the reinforcement.

26. A universal sealing apparatus comprising:
a tape or bandage that comprises:
a first portion configured to removably adhere directly or indirectly to a first outer side of a nose;
a second portion configured to removably adhere directly or indirectly to a second outer side of the nose; and
a third portion between and coupling the first and second portions, the third portion configured to:
removably receive multiple cannulas of a pressurized air-delivery system that are configured to be inserted through the third portion of the universal sealing apparatus and into nostrils of the nose, the cannulas having flared portions, and
place a force upon bases of the flared portions of the cannulas when the first and second portions are adhered to the nose to maintain the flared portions of the cannulas within the nostrils of the nose and to maintain fluid seals of the cannulas with the nose;
wherein the universal sealing apparatus has an elongated shape having (i) first and second longer sides extending along a longer dimension of the universal sealing apparatus and (ii) first and second shorter sides extending along a shorter dimension of the universal sealing apparatus;
wherein the third portion comprises multiple holes between the first and second portions, each hole configured to receive and allow passage of one of the cannulas completely through the third portion;
wherein first and second portions are removably coupleable from the third portion.

27. The apparatus of claim 26, wherein the third portion is divided into two sections that are separated by a rigid angle.

28. The apparatus of claim 26, wherein the tape or bandage has a hinge between the first and second portions of the reinforcement.

29. The apparatus of claim 28, wherein the hinge comprises fabric separating the first and second portions of the reinforcement.

30. The apparatus of claim 28, wherein
the hinge is made of the same materials as the first and second portion of the reinforcement,
the hinge comprises a thinner layer of the material as compared to the first and second portion of the reinforcement.
wherein the reinforcement has a shape with edges that have opposite oblique angles with respect to at least the first longer side of the universal sealing apparatus at opposite ends of the third portion.

31. The apparatus of claim 26, further comprising:
reinforced tips on the outer edges of the first and second portion.

32. The apparatus of claim 26, wherein the first and second portions have portions removed along a side of the longer dimension, the removed portions configured to avoid a touching of the eyes.

33. The apparatus of claim 26, wherein the third portion further comprises a notch configured to accommodate a columella of the nose.
